(19) United States
(12) Patent Application Publication
(10) Pub. No.: US 2022/0167957 A1
(43) Pub. Date: Jun. 2, 2022

(54) Surgical Lingual Retractor Plus

(71) Applicants: Nelson Jose Finol Gutierrez, tampa, FL (US); Margarita Mariela Valdivia Tellez, Tampa, FL (US)

(72) Inventors: Nelson Jose Finol Gutierrez, tampa, FL (US); Margarita Mariela Valdivia Tellez, Tampa, FL (US)

(21) Appl. No.: 17/108,979

(22) Filed: Dec. 1, 2020

(51) Int. Cl. A61B 17/02; A61B 1/06

(52) U.S. Cl. CPC A61B 17/0218; A61B 2217/005; A61B 1/06

(57) ABSTRACT

The Surgical Lingual Retractor Plus device is unique because there are not retractors similar to it, either due to its simplification that allows to retract tissue and protect anatomical structures at the same time, it can fulfill the function of suction of fluids and illumination of the surgical area to be treated by the dental professional or other medical professionals. The protection that it provides to the tissues is contemplated by the fact that it is a device made of stainless steel, providing protection against cutting and high-speed rotating instruments. This device will have a grip component, a retraction component and a protection component connected to a suction and lighting system, all with the purpose of being able to perform all the functions at the same time during the surgery, once simplifies the psychomotor actions by the operator (dental or medical professionals).

### BACKGROUND OF THE INVENTION

The oral cavity is surrounded by folds of skin called lips, inside it are the teeth whose function is to cut and grind food. We also find inside the mouth the tongue considered an organ made up of 8 even muscles and one uneven muscle, taste buds, nerves and blood vessels that provide nerve innervation and vascularization to it.

The oral cavity is constituted by a variety of organs and anatomical structures delimited by themselves, which are innervated by a diversity of nerve endings in a very limited and small space.

Surgical procedures in the oral cavity is a challenge for oral health professionals or specialists. When proceeding to perform surgical interventions that comprise the section or cuts either in hard or soft tissues or the dissection of soft tissues, this raises the possibility of a variety of iatrogenic changes in the area surrounding the surgery in the oral cavity.

Among the most frequent iatrogenic injuries can be found lacerations in the tongue, because this is a very mobile organ, difficult to control or with position limitations during surgical procedures, especially those performed in the lower region of the oral cavity. The tongue has a high degree of mobility that can increase during periods of anxiety, before or during the oral surgical procedure.

Among other iatrogenic that we can highlight are the nervous alterations that are nothing more than alterations that occur at the level of the lingual and alveolar nerves which provide sensory and motor innervation to the tongue, the lower lip and the chin area. These are the areas most injured as a result of surgical treatment in the oral cavity.

Most of these injuries are temporary and can improve in months or can even be permanent depending on the severity of the iatrogenic injury. Damage to these nerves can lead to sensory alterations in the lower lip, tongue and chin region. These conditions can affect the quality of life of the patient which can lead to emotional problems, problems with socialization and disabilities. Accidental injury after surgery can lead to legal claims.

Injuries or complications in patients undergoing dental treatments have become a major social concern that also has often very serious consequences. Most of these injuries could be avoided by retraction and correct positioning of the tongue at the time of the surgical act around this organ, this could be prevented with the use of a “Surgical Lingual Retractor Plus” device that keeps the tongue out of the surgical area.

The patent classification scheme is a system of codes that groups inventions according to technical area, which means similar inventions are grouped under the same classification. Patents are classified as: the design patent, the plant patent and the utility patent.
Design patent: Includes non-obvious, new, original and ornamental designs of manufactured items. They protect only the appearance of the article, not its structural or functional characteristics.
Plant patent: They protect different and new plant varieties reproduced asexually.
Utility patent: It includes the inventions about a new and useful process, machine, article of manufacture, composition or any new useful improvement of the same.

The device that allows positioning the tongue and retracting the tissues in the oral cavity called Surgical Lingual Retractor Plus is contemplated in a utility patent as it is a new device that provides a new utility in the area of dentistry and in related medical-surgical specialties to procedures in the region of the oral cavity.

### BRIEF SUMMARY OF THE INVESTIGATION

Before performing a surgical procedure in the oral cavity, it is important to have an approach condition before activating cutting and rotating instruments, so the retraction and protection of the tissues is important to avoid iatrogenic injuries.

This invention is represented by a surgical device that will allow retraction and protection of the tongue and surrounding tissues, increasing the visualization and the field of work. Therefore, it is important to highlight the following advantages of this device called Surgical Lingual Retractor Plus.

### Advantage

Stable retraction of soft tissues and organs.
Safe protection of hard and soft tissues and organs.
Suction of fluids surrounding the surgical area.
Simplification of psychomotor activities by the professional or assistant of the surgical procedure to be performed.
Autoclavable
Increased visibility due to built-in lighting.
Decrease Aerosol.

### BRIEF DESCRIPTION OF THE SEVERAL VIEWS OF THE DRAWING

FIG. 1: Anterolateral View:

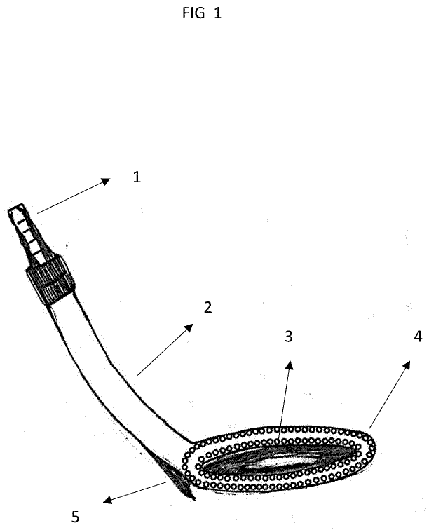

In this view we can visualize five parts of the device.

Part 1: Aspiration anchor, which will connect with the high volume suction of the dental unit

Part 2: Stem, which forms the grip component.

Part 3: Concave part, which is constituted by a smooth surface whose space will be occupied by tissues and organs when performing the retraction maneuver.

Part 4: Multi-perforated edge, made up of multiple 2 mm diameter holes.

Part 5: Led light, which will broaden the vision of the operative field by lighting it, it will have the option of being removed when necessary by the operator.

FIG. 2: Posterolateral

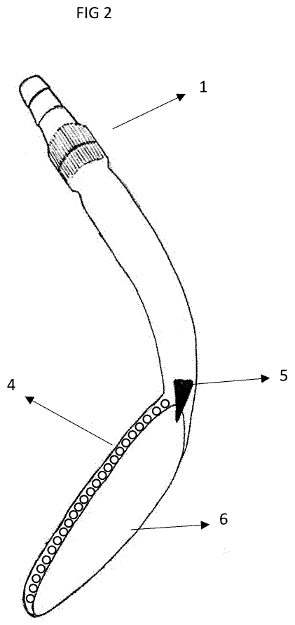

In this view we can visualize four parts of the device.

Part 1: Aspiration anchor, which will connect with the high volume suction of the dental unit

Part 2: Stem, which forms the grip component.

Part 5: Led light, which will broaden the vision of the operative field by lighting it, it will have the option of being removed when necessary by the operator.

Part 6: convex part, consisting of a smooth surface, ovoid in shape and convex throughout its length similar to a spoon, this part constitutes the protection component.

FIG. 3: Anterior View

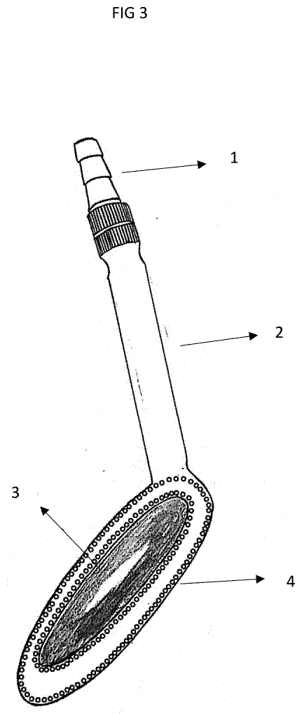

In this view we can visualize four parts of the device.

Part 1: Aspiration anchor, which will connect with the high volume suction of the dental unit

Part 2: Stem, which forms the grip component.

Part 3: Concave part, which is constituted by a smooth surface whose space will be occupied by tissues and organs when performing the retraction maneuver.

Part 4: Multi-perforated edge, made up of multiple 2 mm diameter holes.

### DETAILED DESCRIPTION OF THE INVENTION

The device called Surgical Lingual Retractor Plus will be made of stainless steel in all its structural extension with a spoon shape and will consist of 6 parts: aspiration anchor, steam, concave part, multi-perforated edge, led light and convex part; 3 components: grip component, retraction component and protection component, it will also present an internal suction system with an external lighting system.

Gripping component: Is made up of the steam with one straight end (aspiration anchor) that will be connected to the high volume suction system of the dental chair and with another angled end that will be connected to the retraction component and the protection component. The led light will be located on the posterior area of this component, which will be positioned in the angled area of the stem, close to the front edge of the protection component.

Retraction component: Concave surface allowing the retraction of organs and tissues in that area, it has a multi-perforated edge that will allow the passage of fluids through the activation of the aspiration system.

Protection component: It consists of a convex surface allowing the stabilization and protection of tissues and organs near the surgical area to be intervened.

Next to the union of this component and the gripping component, the lighting system will be fixed, which will allow an increase in the visibility of the surgical area.

It should be noted that this device presents unique factors that make it particular to similar devices which are made of a non-rigid material making it deficient for the protection and stable retraction of tissues and organs in surgical procedures. At the same time, the differentiation is noted with respect to traditional lingual retractors, which do not comply with the multifunctionality of this device (Surgical Lingual Retractor) at the same time.

The multifunctionality of this device are the following: stable retraction and protection of tissues and organs, increased visibility due to lighting and aspiration of fluids, decrease aerosol and simplification of psychomotor maneuvers by the operator and assistant.

1. This device is made of stainless steel with a angled steam attached to a spoon-shaped area with a convex outer smooth surface and a concave inner surface.

2. The convex surface and the concave surface are delimited from each other externally in all their extension by a multi-perforated edge that constitutes part of the suction system internally joined to the rod.

3. Multi-perforated Edge is made up of multiple holes, it has a cylindrical shape and is located on the edge of the device limiting the concave and convex surface.

4. It comprises removable lighting system on the posterior surface of the device, this lighting system is positioned medially next to the junction of the steam and the protection components.

5. The steam has a cylindrical shape with one straight end and one angled end.
